(19) United States
(12) Patent Application Publication
(10) Pub. No.: US 2021/0108165 A1
(43) Pub. Date: Apr. 15, 2021

(54) Organics Removal For Algae Biofuel Systems

(71) Applicant: ExxonMobil Research and Engineering Company, Annandale, NJ (US)

(72) Inventors: Samantha J. Reuter, Houston, TX (US); Rachel G. Munson, Spring, TX (US); Patrick L. Hanks, Bridgewater, NJ (US); Everett J. O'Neal, Asbury, NJ (US); Mark A. Deimund, Jersey City, NJ (US); Sarah E. Feicht, Raritan, NJ (US)

(21) Appl. No.: 17/067,948

(22) Filed: Oct. 12, 2020

Related U.S. Application Data
(60) Provisional application No. 62/913,330, filed on Oct. 10, 2019.

(51) Int. Cl. C12M 1/00; C12N 1/12; C02F 1/32; C02F 1/52; C02F 1/72; C02F 1/78

(52) U.S. Cl. CPC C12M 21/02; C12M 43/02; C12N 1/12; C02F 2101/30; C02F 1/52; C02F 1/722; C02F 1/78; C02F 1/32

(57) ABSTRACT

A system for growing algae for biofuel production includes a bioreactor configured to contain an algae slurry, an algae-water separator fluidly coupled to the bioreactor to receive and separate the algae slurry into algae and separated water, and an organics treatment system that receives a portion of the separated water and is configured to reduce a concentration of organics in the portion of the separated water. A recycle line conveys the portion of the separated water back to the bioreactor following processing in the organics treatment system, wherein the portion of the separated water is recycled and forms part of the algae slurry reducing the raw water demand of the bioreactor.

### CROSS-REFERENCE TO RELATED APPLICATIONS

This application claims the benefit of priority from U.S. Provisional Application No. 62/913,330 filed Oct. 10, 2019, which is herein incorporated by reference in its entirety.

### BACKGROUND OF THE INVENTION

Concerns about climate change, carbon dioxide (CO₂) emissions, and depleting mineral oil and gas resources have led to widespread interest in the production of biofuels from algae, including microalgae. As compared to other plant-based feedstocks, algae have higher CO₂ fixation efficiencies and growth rates, and growing algae can efficiently utilize wastewater, biomass residue, and industrial gases as nutrient sources. Algae are photoautotrophic organisms that can survive, grow, and reproduce with energy derived from the sun through the process of photosynthesis. Photosynthesis is essentially a carbon recycling process through which inorganic CO₂ combines with solar energy, other nutrients, and cellular biochemical processes to output gaseous oxygen and to synthesize carbohydrates and other compounds critical to the life of the algae.

Algae biomass is generally grown in nutrient-containing water within a bioreactor system. Algae bioreactors are sometimes referred to as “photobioreactors” since they utilize a light source to cultivate photoautotrophic algae through photosynthesis. The most common types of bioreactors used in algal cultivation are open raceway ponds and tubular-type enclosed or open reactors.

The bioreactor system mixes and circulates the algae water slurry to ensure adequate exposure to solar energy and gas transfer, thereby promoting the growth of algal biomass. Once the algae matures, various processing methods are employed to separate the algal biomass from the water and extract lipids therefrom for the production of fuel and other oil-based products. The separated water (wastewater) and biomass residue can be recycled or otherwise used in a variety of sustainable applications. For example, the wastewater can be recycled to grow additional algal biomass and the biomass residue can be used as animal feed.

Algae bioreactors require a significant amount of water, so water recycling is imperative for efficient operation. Water recycling, however, can increase the concentration of dissolved organics present in the separated water after a single pass. A buildup of organics could result in operational issues (such as foaming), increased costs at a wastewater treatment plant (WWTP), and/or algae growth inhibition.

### SUMMARY OF THE INVENTION

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In one or more aspects of the disclosure, a system for growing algae for biofuel production is disclosed and includes a bioreactor configured to contain an algae slurry, an algae-water separator fluidly coupled to the bioreactor to receive and separate the algae slurry into algae and separated water, an organics treatment system that receives a portion of the separated water and is configured to reduce a concentration of organics in the portion of the separated water, and a recycle line that conveys the portion of the separated water back to the bioreactor following processing in the organics treatment system, wherein the portion of the separated water forms part of the algae slurry.

In one or more additional aspects of the disclosure, a method of growing algae for biofuel production is disclosed and includes containing an algae slurry within a bioreactor, receiving the algae slurry from the bioreactor at an algae-water separator and separating the algae slurry into algae and separated water with the algae-water separator, receiving a portion of the separated water at an organics treatment system fluidly coupled to the algae-water separator, reducing a concentration of organics in the separated water with the organics treatment system, and conveying the portion of the separated water from the organics treatment system and back to the bioreactor via a recycle line, wherein the portion of the separated water forms part of the algae slurry.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a schematic diagram of an example algae bioreactor that may incorporate the principles of the present disclosure.

FIG. 2 is an example system for growing and harvesting algae for biofuel production.

FIG. 3 is another example system for growing and harvesting algae for biofuel production.

### DETAILED DESCRIPTION OF THE INVENTION

The present disclosure is related to growing algae for biofuel production and, more particularly, to systems and methods for growing algae for biofuel production and including an organics treatment system reduces organics concentration in recycled algae-separated water

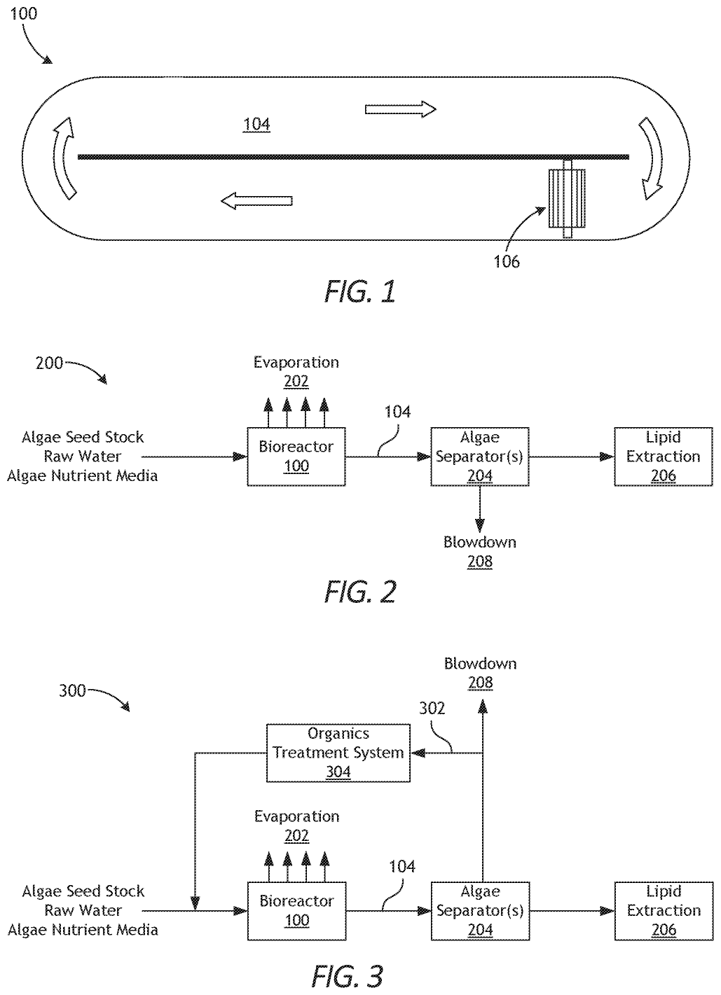

FIG. 1 is a schematic diagram of an example algae bioreactor 100 that may incorporate the principles of the present disclosure. As illustrated, the bioreactor 100 may be a type of “raceway” pond bioreactor that exhibits a generally pill-shaped perimeter and provides a single, closed-loop recirculation channel. The principles of the present disclosure, however, are equally applicable to raceway pond bioreactors exhibiting other geometric shapes, such as circular, ovoid, polygonal (e.g., triangular, square, rectangular, etc.), or any combination thereof. Moreover, the principles of the present disclosure are not limited to raceway pond bioreactors, but are equally applicable to any type of algae bioreactor, such as tubular-type enclosed or open reactors.

The bioreactor 100 is designed to contain an algae slurry 104 for the growth and cultivation of algae. As used herein, the term “algae slurry,” and grammatical variants thereof, refers to a flowable aqueous mixture comprising at least water, algae cells, and algae nutrient media, as discussed in further detail hereinbelow. In some embodiments, the depth of the algae slurry 104 within the bioreactor 100 may be about 12 inches (in.) to facilitate sufficient sunlight penetration needed for algae growth. In other embodiments, however, the depth of the algae slurry 104 may be greater or less than 12 in., without departing from the scope of the disclosure.

Algal sources for the algae growing within the algae slurry 104 can include, but are not limited to, unicellular and multicellular algae. Examples of such algae can include a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species.

In the illustrated embodiment, the bioreactor 100 includes one or more paddlewheels 106 (one shown), or another suitable powered mechanical device, strategically placed in the bioreactor 100 to facilitate continuous or intermittent circulation of the algae slurry 104 within the bioreactor 100. The paddlewheel 106 also operates to mix the algae slurry 104 and thereby help keep the algae properly suspended within the algae slurry 104, which allows the algae to receive sufficient photonic energy from the sun for growth. This also helps prevent sedimentation of the algae cells contained within the algae slurry 104. In other bioreactor designs or configurations, such as in tubular-type enclosed or open reactors, the algae may be circulated and/or mixed continuously or intermittently using a sparging gas injected or recirculated fluid reinjected into the algae slurry 104. The algae slurry 104 circulates and otherwise resides within the bioreactor 100 until the algae contained within the algae slurry 104 matures to a point where lipids may be extracted for biofuel production.

FIG. 2 is an example system 200 that may be used to grow and harvest algae for biofuel production. As illustrated, the system 200 includes the bioreactor 100, which may be fed with raw water to help create the algae slurry 104. In some applications, the raw water may comprise salt water originating from a nearby ocean or another source of salt water. In such embodiments, the salt water may be treated or otherwise filtered to remove substances that might inhibit algae growth in the bioreactor 100. In some embodiments, raw ocean water may be desalinated to provide fresh raw water. In other embodiments, however, the raw water may comprise fresh water obtained from any body or source of fresh water. Moreover, any fresh water sources may be filtered or otherwise treated prior to being introduced into the bioreactor 100.

A prepared algae seed stock may then be added to the raw water in the bioreactor 100, and algae nutrient media may be added to prepare the algae slurry 104 for the cultivation and growth of algae. The algae nutrient media may comprise at least nitrogen (e.g., in the form of ammonia (including ammonium), nitrate, nitrite, or organic compounds containing nitrogen, such as urea) and phosphorous. Other elemental micronutrients may also be included, such as potassium, iron, manganese, copper, zinc, molybdenum, vanadium, boron, chloride, cobalt, silicon, and the like, and any combination thereof. The order in which the algae seed stock and the algae nutrient media are added to the raw water in the bioreactor 100 is not critical and either may be added before the other or they may be added simultaneously, without departing from the scope of the present disclosure.

The algae slurry 104 may reside in the bioreactor 100 for a predetermined amount of time or until the algae matures and is ready for harvesting. Typical residence time in the bioreactor 100 can range between about 2 days and about 20 days. While the algae cultivates and grows within the bioreactor 100, the algae slurry 104 will commonly lose a portion of the raw water in the form of evaporation 202. Once the algae matures and is otherwise ready for harvesting, the algae slurry 104 is extracted from the bioreactor 100 and pumped to one or more algae-water separators 204 to be harvested and dewatered, during which the algae in algae slurry 104 is generally separated from the water. The algae-water separator(s) 204 may comprise any known separator, filter, or dewatering system known, and can include any combination thereof.

The separated algae is then conveyed downstream for lipid extraction 206 in preparation for biofuel production. In the illustrated embodiment, the separated water can be purged from system 200 via a blowdown stream 208 and discharged into the environment or reused for another application. In some cases, the separated water purged via the blowdown stream 208 is conveyed to a wastewater treatment plant for treatment so that the separated water can be discharged into the environment with minimal impact.

As will be appreciated, system 200 uses a large amount of raw water to cultivate, grow, and harvest the algae for biofuel production. In one example operation of the system 200, for instance, about 84 million gallons per day (MGD) of raw water is provided to the bioreactor 100 to generate the algae slurry 104. About 38 MGD of the raw water evaporates 202 from the algae slurry 104 during algae growth and cultivation. Consequently, the algae slurry 104 extracted from the bioreactor 100 comprises about 46 MGD when pumped to the algae-water separator(s) 204. Following harvesting and dewatering of the algae from the algae slurry 104 within the algae-water separator(s) 204, about 2 MGD of water accompanies the separated and concentrated algae for lipid extraction 206. The remaining separated water comprises about 44 MGD and is purged from the system 200 via the blowdown stream 208. Consequently, growing, harvesting, and obtaining algae for lipid extraction in the system 200 requires a significant amount of raw water that must be replaced following each harvesting cycle.

FIG. 3 is another example system 300 for growing and harvesting algae for biofuel production. As described herein, the system 300 may incorporate one or more principles of the present disclosure. The system 300 may be similar in some respects to the system 200 and, therefore, may be best understood with reference thereto, where like numerals will correspond to like components or elements not described again in detail. As illustrated, the system 300 may include the bioreactor 100, which may be fed with raw water, algae seed stock, and algae nutrients media to generate the algae slurry 104 suitable for the cultivation and growth of algae. While the algae grows within the bioreactor 100, the algae slurry 104 may lose a portion of the raw water to evaporation 202, as discussed above. Once the algae matures and is ready for harvesting, the algae slurry 104 is extracted from the bioreactor 100 and pumped to the algae-water separator(s) 204 to be harvested and dewatered. The algae separated from the algae slurry 104 may then be conveyed downstream for lipid extraction 206 in preparation for biofuel production.

In the illustrated embodiment, the system 300 may further include a recycle conduit or line 302 fluidly coupled to the algae-water separator(s) 204 to receive the separated water after harvesting. Some of the separated water may be conveyed to the recycle line 302 while the rest of the separated water may be purged from the system 300 via the blowdown stream 208. The recycle line 302 may be configured to convey the separated water back to the bioreactor 100 to be reused in the creation of another batch of the algae slurry 104 for a subsequent cycle of algae growth and cultivation. In some cases, the recycled separated water will provide the majority (e.g., 50% or more) of the raw supply water used to make new algae slurry 104. Accordingly, recycling the separated water reduces the raw water demand of the bioreactor 100 while simultaneously reducing the amount of water purged via the blowdown stream 208 and requiring treatment prior to environmental discharge.

In one example operation of the system 300, the algae slurry 104 in the bioreactor 100 may comprise about 42 MGD of the raw water and about 42 MGD of the recycled separated water, thus providing about 84 MGD of water to generate the algae slurry 104. About 38 MGD of the water evaporates 202 from the algae slurry 104 during algae growth and cultivation. Consequently, the algae slurry 104 extracted from the bioreactor 100 comprises about 46 MGD when pumped to the algae-water separator(s) 204 to be harvested and dewatered. Following harvesting and dewatering in the algae-water separator(s) 204, about 2 MGD of water from the algae slurry 104 accompanies the separated and concentrated algae for lipid extraction 206, while the separated water discharged from the algae-water separator(s) 204 may comprise about 44 MGD. Of the 44 MGD of separated water, about 2 MGD may be purged via the blowdown stream 208, and the remaining 42 MGD of the separated water may again be recycled back to the bioreactor 100 via the recycle line 302 to generate a new batch of the algae slurry 104.

While recycling the separated water back to the bioreactor 100 reduces the raw water demand of the bioreactor 100, the recycled separated water will exhibit an increased concentration of dissolved organics after only a single pass (cycle) through the system 300. For example, the amount of dissolved organic material present in the separated water after one cycle through the system 300 may be about 50 to about 100 milligrams per liter (mg/L) of water. Cycling the separated water through the system 300 a second time will further increase the concentration of dissolved organic material. The continued increase in concentration of organics in the water used for the algae slurry 104 will eventually reach a threshold limit beyond which the recycled water will create problems and inhibit algae growth and/or lipid accumulation. Elevated concentrations of organics, for example, can result in operational issues (e.g., foaming) and increased costs of treating separated water at wastewater treatment plants.

According to embodiments of the present disclosure, the system 300 may further include an organics treatment system 304 configured to receive and process the separated water to reduce the concentration of organics prior to being recycled back to the bioreactor 100. In some applications, for example, the separated water may enter the organics treatment system 304 with a dissolved organic material concentration of about 50 to about 100 mg/L, and after being processed in the organics treatment system 304, the concentration of dissolved organic material in the separated water may be reduced to about 0 mg/L. As will be appreciated, operation of the organics treatment system 304 is not limited to the foregoing example, but can instead be used to receive and process the separated water of any influent organics concentration, without departing from the scope of the disclosure.

The organics treatment system 304 may comprise any type of process or system operable to reduce the concentration of organics in the separated water and discharge recycled water with reduced organics or reduced organics concentration. In some embodiments, the organics treatment system 304 may comprise a chemical oxidation process or system configured to oxidize the incoming separated water. This oxidation process reduces the buildup and concentration of organics by converting some fraction of the organics to carbon dioxide (CO₂). The remaining fraction of the organics that does not convert to CO₂ may transition from larger organic molecules to smaller, potentially less disruptive organic molecules. The smaller organic molecules will act less like surfactants for foaming or be less toxic or inhibitory to the growth of algae.

The chemical oxidation system can have different configurations, depending on what oxidants are used to oxidize the separated water. In some embodiments, for example, the chemical oxidation system may have a reaction chamber that receives the separated water and into which oxidants are introduced to oxidize the separated water. Example oxidants that may be used in the chemical oxidation system include, but are not limited to, ozone (O₃), a peroxide (e.g., hydrogen peroxide or H₂O₂), ultraviolet (UV) light, photocatalytic oxidation, or any combination thereof. The dose and contact time requirements for the oxidants will be designed based on the types of soluble organics present within the separated water. Moreover, the designed maximum soluble organics concentration allowable in the separated water discharged from the chemical oxidation system will be controlled by final biology and operations and will likely be site dependent.

In other embodiments, the organics treatment system 304 may comprise a chemical coagulation process that may include flocculation. Chemical coagulation is a process that destabilizes the surface charge of particulate, colloidal, and/or dissolved organics allowing the organics to aggregate together or attach to other solids present in solution. Flocculants may be added after coagulation to further aggregate solids to make solids-liquid separation easier. Example chemical coagulants and flocculants that may be used include, but are not limited to, organic and inorganic blended coagulants and organic flocculants, or any combination thereof. Following coagulation and flocculation, one or more physical separation techniques may be used to remove the organics out of the separated water. Example physical separation techniques include, but are not limited to, gravity separation (i.e., clarification), granular media filtration, membrane filtration, or any combination thereof.

In yet other embodiments, the organics treatment system 304 may comprise a filtration process or system that physically separates and removes particulate or colloidal organics. In such embodiments, the algae-water separator 204 may comprise a system that does not use membrane filtration, which would mitigate or eliminate particulate, colloidal, and dissolved organics present within the slurry. Depending on the pore size, membrane filters will typically remove the particulate and some amount of the colloidal organics. In even further embodiments, the organics treatment system 304 may comprise a combination of any of the foregoing processes or systems, without departing from the scope of the disclosure.

As will be appreciated, removing dissolved organics from the separated water may also decrease the amount of water that must be purged via the blowdown stream 208. More specifically, to ensure that the recycled separated water does not surpass predetermined concentrations of organics, a portion of the separated water is commonly purged from the system 300 via the blowdown stream 208. The volume of water lost to the blowdown stream 208 is then replenished in the bioreactor 100 with fresh raw water. The purge rate through the blowdown stream 208 is sometimes determined by organic build-up in the system 300, and removing the dissolved organics from the separated water helps decrease the amount of water that must be purged via the blowdown stream 208.

Moreover, the organics treatment system 304 may help reduce the algae nutrient media demand. More particularly, in prior systems, large portions of the separated water would be purged via the blowdown stream 208 to help maintain low levels of organics within the bioreactor 100. All nutrients contained in the separated water would be purged along with the water. With the organics treatment system 304, however, the concentration of dissolved organics is reduced and the algae nutrient media (e.g., nitrogen, sulfur, and carbon) present in the separated water discharged from the organics treatment system 304 can be consumed by the algae in the bioreactor 100 upon being recycled. Consequently, those raw nutrients can be recycled and used to grow algae instead of being purged from the system 300 by the blowdown stream 208. All recycled nutrients directly reduce the fresh algae nutrient media demand.

### Embodiments Listing

The present disclosure provides, among others, the following embodiments, each of which may be considered as optionally including any alternate embodiments.

Clause 1. A system for growing algae for biofuel production including a bioreactor configured to contain an algae slurry, an algae-water separator fluidly coupled to the bioreactor to receive and separate the algae slurry into algae and separated water, an organics treatment system that receives a portion of the separated water and is configured to reduce a concentration of organics in the portion of the separated water, and a recycle line that conveys the portion of the separated water back to the bioreactor following processing in the organics treatment system, wherein the portion of the separated water forms part of the algae slurry.

Clause 2. The system of Clause 1, wherein the portion of the separated water comprises a first portion and the system further comprises a blowdown stream fluidly coupled to the algae-water separator to receive a second portion of the separated water to be purged from the system.

Clause 3. The system of Clause 2, wherein the first portion of the separated water comprises a majority of the separated water.

Clause 4. The system of any of the preceding Clauses, wherein the organics treatment system comprises a chemical oxidation system that oxidizes the portion of the separated water.

Clause 5. The system of Clause 4, wherein the chemical oxidation system converts a first fraction of the organics to carbon dioxide, while a second fraction of organics transitions from larger organic molecules to smaller organic molecules.

Clause 6. The system of Clause 4, wherein the chemical oxidation system uses an oxidant selected from the group consisting of ozone, a peroxide, ultraviolet light, a photocatalytic oxidation, and any combination thereof.

Clause 7. The system of any of Clauses 1 to 3, wherein the organics treatment system comprises a chemical coagulation process that uses a chemical coagulant to pull the organics out of solution in the portion of the separated water.

Clause 8. The system of Clause 7, wherein the chemical coagulant is selected from the group consisting of an organic blended coagulant, an inorganic blended coagulant, an organic flocculant, and any combination thereof.

Clause 9. The system of Clause 7, wherein the chemical coagulation process includes a physical separation technique used to remove organics out of the portion of the separated water.

Clause 10. The system of any of Clauses 1 to 3, wherein the organics treatment system comprises a filtration system that physically separates and removes the organics from the portion of the separated water.

Clause 11. The system of any of the preceding Clauses, wherein the bioreactor is selected from the group consisting of a raceway pond bioreactor, a tubular-type enclosed bioreactor, a tubular-type open bioreactor, and any combination thereof.

Clause 12. The system of any of the preceding Clauses, wherein the algae-water separator is selected from the group consisting of a fluid separator, a filter, a dewatering system, and any combination thereof.

Clause 13. A method of growing algae for biofuel production includes containing an algae slurry within a bioreactor, receiving the algae slurry from the bioreactor at an algae-water separator and separating the algae slurry into algae and separated water with the algae-water separator, receiving a portion of the separated water at an organics treatment system fluidly coupled to the algae-water separator, reducing a concentration of organics in the separated water with the organics treatment system, and conveying the portion of the separated water from the organics treatment system and back to the bioreactor via a recycle line, wherein the portion of the separated water forms part of the algae slurry.

Clause 14. The method of Clause 13, wherein the portion of the separated water comprises a first portion and the method further comprises purging a second portion of the separated water received from the algae-water separator via a blowdown stream.

Clause 15. The method of Clause 14, wherein the first portion of the separated water comprises a majority of the separated water.

Clause 16. The method of any of Clauses 13 to 15, wherein the organics treatment system comprises a chemical oxidation system, the method further comprising oxidizing the portion of the separated water and thereby converting a first fraction of the organics to carbon dioxide and transitioning a second fraction of organics from larger organic molecules to smaller organic molecules.

Clause 17. The method of Clause 16, wherein the chemical oxidation system uses an oxidant selected from the group consisting of ozone, a peroxide, ultraviolet light, photocatalytic oxidation, and any combination thereof.

Clause 18. The method of any one of Clauses 13 to 15, wherein the organics treatment system comprises a chemical coagulation process comprising adding a chemical coagulant to the portion of the separated water, pulling dissolved organics out of solution in the portion of the separated water with the chemical coagulant and thereby obtaining solid organics, and removing the solid organics from the portion of the separated water.

Clause 19. The method of any one of Clauses 13 to 15, wherein the organics treatment system comprises a filtration system and the method further comprises physically separating and removing the organics from the portion of the separated water with the filtration system.

Clause 20. The method of any one of Clauses 13 to 19, further comprising providing greater than 50% of required water for the algae slurry with the portion of the separated water conveyed from the organics treatment system.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The invention claimed is:

1. A system for growing algae for biofuel production, comprising:
a bioreactor configured to contain an algae slurry;
an algae-water separator fluidly coupled to the bioreactor to receive and separate the algae slurry into algae and separated water;
an organics treatment system that receives a portion of the separated water and is configured to reduce a concentration of organics in the portion of the separated water; and
a recycle line that conveys the portion of the separated water back to the bioreactor following processing in the organics treatment system, wherein the portion of the separated water forms part of the algae slurry.

2. The system of claim 1, wherein the portion of the separated water comprises a first portion and the system further comprises a blowdown stream fluidly coupled to the algae-water separator to receive a second portion of the separated water to be purged from the system.

3. The system of claim 2, wherein the first portion of the separated water comprises a majority of the separated water.

4. The system of claim 1, wherein the organics treatment system comprises a chemical oxidation system that oxidizes the portion of the separated water.

5. The system of claim 4, wherein the chemical oxidation system converts a first fraction of the organics to carbon dioxide, while a second fraction of organics transitions from larger organic molecules to smaller organic molecules.

6. The system of claim 4, wherein the chemical oxidation system uses an oxidant selected from the group consisting of ozone, a peroxide, ultraviolet light, a photocatalytic oxidation, and any combination thereof.

7. The system of claim 1, wherein the organics treatment system comprises a chemical coagulation process that uses a chemical coagulant to pull the organics out of solution in the portion of the separated water.

8. The system of claim 7, wherein the chemical coagulant is selected from the group consisting of an organic blended coagulant, an inorganic blended coagulant, an organic flocculant, and any combination thereof.

9. The system of claim 7, wherein the chemical coagulation process includes a physical separation technique used to remove organics out of the portion of the separated water.

10. The system of claim 1, wherein the organics treatment system comprises a filtration system that physically separates and removes the organics from the portion of the separated water.

11. The system of claim 1, wherein the bioreactor is selected from the group consisting of a raceway pond bioreactor, a tubular-type enclosed bioreactor, a tubular-type open bioreactor, and any combination thereof.

12. The system of claim 1, wherein the algae-water separator is selected from the group consisting of a fluid separator, a filter, a dewatering system, and any combination thereof.

13. A method of growing algae for biofuel production, comprising:
containing an algae slurry within a bioreactor;
receiving the algae slurry from the bioreactor at an algae-water separator and separating the algae slurry into algae and separated water with the algae-water separator;
receiving a portion of the separated water at an organics treatment system fluidly coupled to the algae-water separator;
reducing a concentration of organics in the separated water with the organics treatment system; and
conveying the portion of the separated water from the organics treatment system and back to the bioreactor via a recycle line,
wherein the portion of the separated water forms part of the algae slurry.

14. The method of claim 13, wherein the portion of the separated water comprises a first portion and the method further comprises purging a second portion of the separated water received from the algae-water separator via a blowdown stream.

15. The method of claim 14, wherein the first portion of the separated water comprises a majority of the separated water.

16. The method of claim 13, wherein the organics treatment system comprises a chemical oxidation system, the method further comprising oxidizing the portion of the separated water and thereby converting a first fraction of the organics to carbon dioxide and transitioning a second fraction of organics from larger organic molecules to smaller organic molecules.

17. The method of claim 16, wherein the chemical oxidation system uses an oxidant selected from the group consisting of ozone, a peroxide, ultraviolet light, photocatalytic oxidation, and any combination thereof.

18. The method of claim 13, wherein the organics treatment system comprises a chemical coagulation process comprising:
adding a chemical coagulant to the portion of the separated water;
pulling dissolved organics out of solution in the portion of the separated water with the chemical coagulant and thereby obtaining solid organics; and
removing the solid organics from the portion of the separated water.

19. The method of claim 13, wherein the organics treatment system comprises a filtration system and the method further comprises physically separating and removing the organics from the portion of the separated water with the filtration system.

20. The method of claim 13, further comprising providing greater than 50% of required water for the algae slurry with the portion of the separated water conveyed from the organics treatment system.
